(19) United States
(12) Patent Application Publication
(10) Pub. No.: US 2010/0305476 A1
(43) Pub. Date: Dec. 2, 2010

(54) GUIDEWIRE SENSOR DEVICE AND SYSTEM

(75) Inventors: Paul P. Thornton, Galway (IE); Thomas J. Gaskin, Galway (IE); Finbar Dolan, Moate (IE)

Correspondence Address: CHRIS A. CASEIRO, VERRILL DANA, LLP, ONE PORTLAND SQUARE, PORTLAND, ME, 04112-0586, US

(73) Assignee: ULBRICH PRECISION METALS LIMITED, Oranmore (IE)

(21) Appl. No.: 12/786,849

(22) Filed: May 25, 2010

(30) Foreign Application Priority Data
May 28, 2009 (IE) S2009/0420

(51) Int. Cl. A61M 25/09

(52) U.S. Cl. 600/585

(57) ABSTRACT

The present invention provides a guidewire device for determining or measuring biological, physical or topographical data at a site within a lumen duct or pipe in medical and non-medical applications. Guidewire device 1 includes one or more sensor device 10. Sensor device 10 is integrally mounted on a guidewire device adjacent a distal tip of the guidewire 1. Data gathered by the sensor device 10 when actuated is converted into an electromagnetic output that is wirelessly transmitted via an input/output system between the sensor device and an external electronic device 20.

### BACKGROUND OF THE INVENTION

1. Field of the Invention

The present invention relates to guidewires and in particular to guidewires used in medical diagnostic or treatment applications and to other diverse non-medical applications which call for or benefit from use of a guidewire and sensor. Priority is claimed from Irish Patent Application No. S2009/0420 dated 28 May 2009. The entirety of that priority application is incorporated herein by reference.

2. Description of the Prior Art

Many diverse procedures call for manipulation or visualisation at a remote, inaccessible location and typically a guidewire is used to facilitate the positioning of a device at the desired part of the inaccessible location.

Guidewires have particular application in minimally invasive medical procedures where they are used to guide catheters or other medical devices to a target site within the human or animal body. The guidewire is typically advanced to a desired target site, such as a diagnosis or treatment site, for example the site of a lesion or blockage in a vein or artery or other body lumen. Other interventional medical devices, such as guide catheters, therapeutic catheters, or diagnostic catheters, are introduced over or along the guidewire and directed through sometimes tortuous vasculature to the site of the arterial or venour or other blockage, lesion or treatment site.

Guidewires are used in minimally invasive percutaneous transluminal coronary angioplasty (PTCA) and peripheral angioplasty procedures. In PTCA procedures, a guidewire is typically inserted into the femoral artery of a patient near the groin, advanced over the aortic arch, through a coronary ostium and into a coronary artery to a target site. A guidewire insertion procedure is typically performed in a hospital setting using fluoroscopy to visualise and assist the advancement of the guidewire to the desired target site.

Other medical uses of guidewires include, without limitation, use in urinary, gastro-intestinal, pulmonary and bilary applications. Guidewires are also used in other interventional, investigative and surgical applications and procedures.

As mentioned above, the principle function of the known guidewire is to facilitate access to a remote location within a body lumen, thereby making the location of the site available to adjuvant diagnostic or treatment devices. This is achieved by providing the guidewire as a flexible wire which is capable of traversing bodily channels, vessels or passageways (generally referred to as lumens) which are often extremely tortuous. The guidewire is sufficiently flexible to be navigable to the desired site without damaging the walls of the vessels it passes through. Also as mentioned above, once the guidewire has been positioned at the desired location, it is used as a guide over or along which other devices can be accurately guided to or placed at the target site. For example, in balloon angioplasty, a catheter having a balloon at its distal tip is introduced, using the guidewire to guide it, to an area of a blood vessel which is blocked or partially blocked by atherosclerotic deposits (plaque). The atherosclerotic deposits on the vessel wall reduce or prevent blood flow. The balloon is inflated at the site of the plaque to compress the plaque against the vessel wall thereby opening the blocked or partially blocked vessel to the flow of blood. In a related procedure, a stent may be advanced over or along the guidewire to the site of blockage and deployed there to provide an artificial scaffolding which maintains the vessel in the open state in which good blood flow occurs.

The term “distal” as used herein refers to that extremity of a guidewire device that leads as the device is introduced into a body. Thus, “distal” may be thought of as “distal” from the operator deploying the device. By contrast, the term “proximal” as used herein refers to a portion of the device which trails the distal portion as the device is introduced into a body. Thus “proximal” may be thought of as “proximal” to an operator.

Implantable devices such as stents have been described as a means for introducing a sensor device to a desired site within the body. U.S. Pat. No. 6,729,336 describes an implantable stent which is adapted to enable the physician to detect whether restenosis is occurring. Restenosis is a complication which occurs in a significant number of patients when for various reasons, re-epithelialisation within an implanted stent occurs causing the vessel to again become narrowed or blocked, leading to ischemia, angina or myocardial infarction if left untreated. The stent of U.S. Pat. No. 6,729,336 includes in its make-up a sensor device which can measure blood flow and which can be activated non-invasively externally of the patient using electromagnetic energy. Data detected by the sensor on activation can also be measured using an ultrasound detector externally of the patient.

U.S. Pat. No. 5,411,551 describes an implantable stent of the type which is biased into an expanded state and is contracted for delivery. It includes on its inner surface a recess for retaining a sensor device for measuring, in particular, blood glucose level.

### SUMMARY OF THE INVENTION

In many applications, there will be no implanted device present to act as a sensor. For such applications, there is a need for a device which can gather and retrieve desired data without need of an implant.

Accordingly, the present invention provides a device comprising a guidewire and a wireless electromagnetic (EM) energy enabled integral sensor.

Ideally the sensor is radio-frequency (RE) enabled. The guidewire comprises an antenna for transmissions of wireless signals to and from the sensor.

Preferably, the guidewire is adapted to act as an antenna.

The sensor may be printed, etched or micro-machined onto the surface of the guidewire. In a preferred arrangement, the sensor is disposed at or near the distal tip of the guidewire.

Various kinds of sensors are included within the scope of the invention. For example, the sensor may comprise a sensor for measuring a biological variable. Such a sensor includes, without limitation, a sensor for detecting chemical variables; biochemical variables; pH; oxygenation levels; proteins; antigens or antibiotics; and bio-markers. The sensor may also comprise a sensor for detecting a physical variable such as flow rate, temperature or pressure. The structural integrity or topology of a structure may be assessed or determined by the choice of a sensor usable for such determination.

Power means are provided on the sensor for energising the sensor in response to a remotely-generated EM signal transmitted to the sensor by wireless means. In one arrangement the power means comprises a mechanical resonant member.

Ideally, the sensor includes an electronic processor including, a wireless receiver for receiving a remotely-generated actuation signal and a wireless transmitter for transmitting sensed data to an external device. Preferably, the processor includes memory means for storing sensed data.

In a preferred arrangement, the electronic processor transforms the sensed data into a modulated form for wireless transmissions to an external device.

A guidewire may be provided with a plurality of sensors for measuring different desired data.

The present invention also provides a system comprising a guidewire including a sensor device as described above and at least one external electronic device which communicates wirelessly with the guidewire sensor to activate the sensor and/or to receive sensed data transmitted from the guidewire sensor.

In a preferred arrangement, the external electronic device includes means for processing data received from the sensor and converting it into a visual, textual or audio output. In one embodiment, the external electronic device uses surface acoustic wave (SAW) technology to process or convert received data.

The external device is used to excite a sensor and capture attenuated acoustic waves reflected from a surface. The nature of the surface and how it is altered, as well as the media through which the waves travel, attenuate the signals in a manner which is characteristic of the materials in contact with the surface. Typically, the sensor will include a piezoelectric substrate with a standard arrangement of input and output transducers. The surface may additionally or alternatively include a sensor coating suitable for the particular feature to be measured.

Each guidewire sensor device may include a plurality of different sensors, and the external electronic device is adapted to process and/or convert data from each sensor, individually or collectively. Ideally, the external electronic device both transmits an actuating signal to the or each sensor and receives data from the or each sensor. Where a plurality of sensors are disposed on a guidewire, they may be interrogated singly, collectively or in any desired combination of individual sensors and may be arranged to transmit correspondingly.

The external electronic device may optionally be a handheld device.

In another aspect, the invention includes a method for detecting desired data comprising:

providing a guidewire sensor as described above;

providing an external electronic device adapted to communicate by wireless means with the guidewire sensor;

generating and transmitting a wireless electromagnetic signal from the external electronic device capable of actuating the guidewire sensor;

receiving the generated signal at the guidewire sensor and processing the signal to activate the sensor to gather data;

optionally storing sensed data in a memory means within the sensor;

optionally modulating the sensed data within the sensor;

transmitting the sensed data by wireless means to the external electronic device; and

processing data received by the external electronic device; and optionally providing a data output.

In a further aspect, the invention provides for the use of the guidewire sensor, or system, or method of the present invention in a medical, therapeutic, diagnostic or intervention application. In another aspect, the invention provides for the use of the guidewire sensor, or system, or method of the present invention in non-medical applications, including but not limited to monitoring or maintenance of fuel lines, water lines and the like.

### BRIEF DESCRIPTION OF THE DRAWINGS

The invention will now be described with reference to the accompanying drawings, which show by way of example only, embodiments of a guidewire sensor device according to the invention and in which;

FIG. 1 is a cross-sectional view of the distal tip of a guidewire according to the invention;

FIG. 2 is a view of a part of the distal tip of a guidewire carrying a micromachined sensor array;

FIG. 3 is a view of a part of the distal tip of a guidewire carrying an etched sensor with a chamber for receiving a biomedical device; and

FIG. 4 is a schematic view of a system incorporating the guidewire of the invention together with an external electronic device.

### DETAILED DESCRIPTION OF THE INVENTION

The guidewire of the invention has applications in several different manners and the guidewire will be constructed and arranged appropriately, depending on the desired application. It can be used to integrate wireless communications with stand-alone sensor technology. It has usefulness in the integration of surface acoustic wave (SAW) wireless RF sensors at the distal end of a guidewire device.

Known guidewire technology can be used in the invention to construct the guidewire sensor of the invention. Typically, such known guidewires are constructed as a coil of wire. Guidewire needs to be very flexible in order to be able to pass through often tortuous body passageways to a desired target site. Coils have been found to be well suited for this purpose since the coils, together with the selection of a suitable wire material, lend the advantages of good flexibility combined with necessary strength. Many guidewires have a leading (distal), tip portion comprising a coil which is even more flexible than the main body-length of the guidewire. This is to provide added flexibility to assist the tip in passing or traversing contorsions in a vessel.

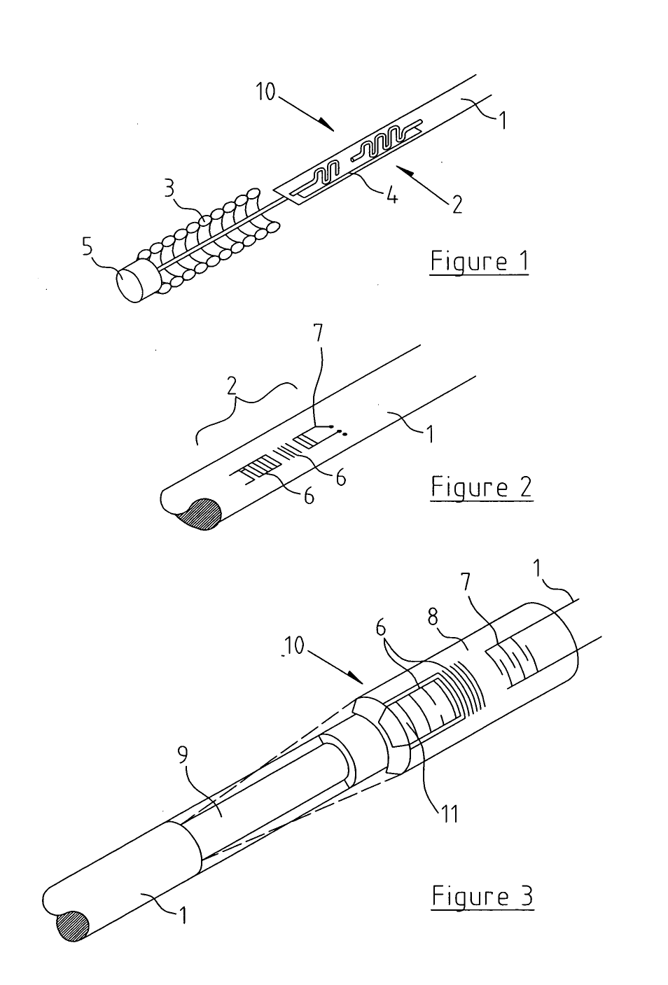

FIG. 1 shows a cross-sectional view of a guidewire device according to the invention. Guidewire 1 includes a distal tip 2 which includes a flexible additional leading coil 3 at the distal tips. Mounted on the distal extremity of the tip is an oximeter 5 for measuring the oxygen saturation of the blood flowing past the distal tip of the guidewire 1. Also mounted at the distal tip but proximal of the oximeter 5 is a sensor 10 including an RF quartz crystal micro balanced sensor 4. The guidewire 1 acts as an antenna. Included as part of the sensor device 10 is an input/output IDT (interdigital transducer) switch.

FIG. 2 shows a view of the distal tip of another embodiment of a guidewire device according to the invention. In this case, guidewire 1 carries at its distal tip a sensor 10 carrying an input/output IDT switch 7 and an acoustic reflector array 6 machined onto the guidewire. The reflector array acts as the “transmission” lines in the device and provides the means for operation of the circuit.

FIG. 3 shows an embodiment of a guidewire according to the invention with an RF sensor device 10 comprising an insert 8 carrying an input/output IDT switch 7 and an acoustic reflector array 6, both mounted onto the insert 8. The insert 8 is shown disassembled from the guidewire. A cavity 9 is etched into the guidewire 1 and upon assembly insert 8 is located into the etched cavity 9. This device includes a biosensor inlet 11 which provides access for bodily fluid to enter a chamber within the sensor in which any desired biochemical or diagnostic test or measurement occurs upon an energisation of the sensor. Data gathered by the sensor is converted into an electromagnetic signal output for the input/output switch. The device may have surface-printed molecular entities that conjugate or interact with an analyte of interest. One or more channels or other structures may be etched or machined into the surface to ensure that sufficient analyte sample is captured.

In all the embodiments shown, the guidewire itself acts as the antenna for receiving and transmitting RF energy between the sensor device on the guidewire and an external electronic device.

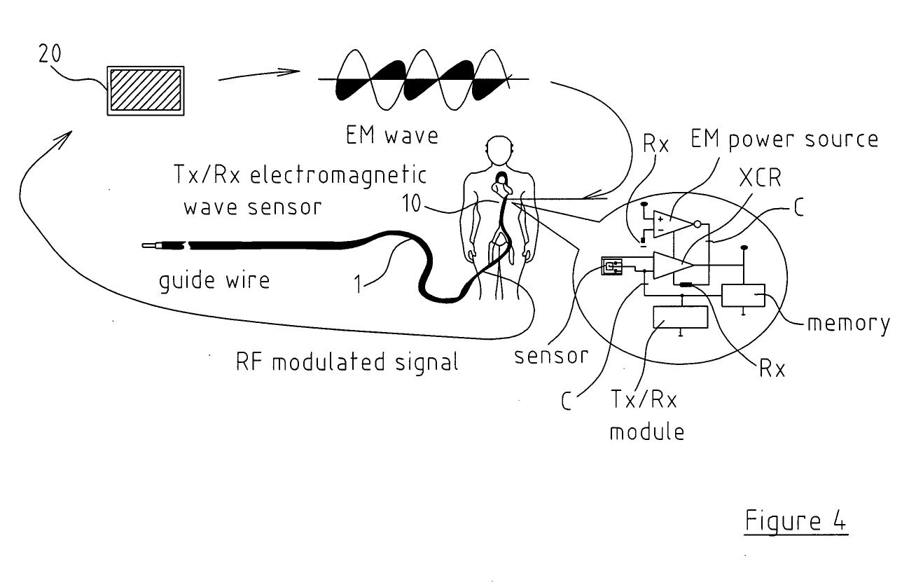

FIG. 4 is an overview of how the guidewire of the invention is operated in use. Included in the figure is a circuit diagram outlining the operation of the circuitry of the sensor device. A guidewire 1 carrying an appropriate sensor for the desired application is introduced into the body of a patient, in this example, to the coronary vasculature. Any well known techniques, such as fluoroscopy, is used to guide the distal tip of the guidewire into the desired location. Once the guidewire is properly positioned, the sensor is activated by an RF signal generated by and transmitted from an external electronic device 20, which may optionally be a handheld device. The RE signal is received by the input/output IDT switch of the sensor 10 with a length of the guidewire acting as an antenna. A standard piezoelectric circuit is suitable for this purpose. The electromagnetic RE signal received by the sensor 10 activates it so that it carries out its pre-determined sensor function. The data collected by the sensor 10 is then converted to an output electromagnetic signal and transmitted as a modulated RF signal back to the external device 20. Optionally, a memory means is provided in the circuit to allow data gathered by the sensor to be stored until an interrogation signal is received from the external device 20, at which time the data stored in the sensor memory is transmitted to the external device. Data received from the sensor 10 by the external device 20 is then handled in conventional ways, for example by processing to provide visual, audio, text or other outputs which are meaningful to the clinician.

The present invention provides a guidewire which has integrated, at its distal tip, a system for measuring, in vivo, physiological and biochemical characteristics of bodily lumens or fluids passing through such lumens, such as but not limited to the arterial and venous circulatory systems. Integration of the sensors with wireless technology enables remote characterisation of various anatomical and physiological variables of coronary and peripheral arterial and venous systems, neural, pulmonary and bilary systems, as well as other anatomies.

The present invention provides a conventionally designed guidewire upon which is placed a single sensor or an array of sensors at the distal end of the guidewire. Such sensors are selected to be suitable to characterise a target datum such as arterial or venous blood vessels and to measure physiological variables (cellular integrity etc.) in vivo.

The sensor includes a transducer and a wireless transmitter/receiver (e.g., radio frequency RE) and the guidewire itself acts as the antenna. Sensors are selected to suit the specific variable or variables to be measured and adapted to be sensitive to relevant changes in response to the physiological variable being measured. Measured data is optionally transferred into memory and can be transmitted in real time or from memory to an external electronic device (such as e.g. RF enabled device). Upon request, the external device serves two primary functions; the first is to broadcast an electromagnetic signal that excites the sensor into action (e.g. using a mechanical resonant member); the second is to receive an attenuated signal generated in and broadcast from an RF tag located on the sensor device, which it monitors and translates into an output which is convenient for an operator. The sensor output is reduced into a mathematical linear or quadratic relationship with the physiological variable measured and variances are, identified and interpreted by software embedded, usually, in the external electronic device. In a preferred arrangement, an output reading is offered to the operator which provides easily readable interpretation of the measured characteristics. The response data from the sensor may also be stored in memory in the sensor housing at the distal tip of the guidewire.

In a preferred arrangement, the sensor comprises a mechanical resonance member which can be excited into a resonant state by an electromagnetic signal generated and transmitted by the electronic device which is located external to the patient's body. The sensor or sensors have the ability to respond to resonant excitation from the external electronic device. Further, the sensor has the ability to transmit the response data to the external electronic device during or post resonant excitation via an RF modulated signal. The external electronic device demodulates the RF signal from the sensor in vivo. Is also performs a signal analysis of the data from the sensor or sensors on the guidewire in vivo.

In an alternative arrangement, the sensor may include a power means, such as a battery, which is switchable between on and off states by an electromagnetic signal generated by the external device. The electromagnetic signal may be any frequency which is detectable and is usefully an RF signal.

The guidewire ideally incorporates one or more preferably integral sensors that can be excited and interrogated using an RF transmitter/receiver in an electronic device which is located externally, and which may be a fixed device or a handheld device.

Powering up the sensor is achieved through electromagnetic excitation (ideally using RF energy) by the electronic device 20 external to the patient's body. Once powered up, the sensor transmits the physiological variable information it senses or has stored, for example, blood flow, blood oxygen saturation levels, temperature, pH, blood chemistry, the presence of protein markers, the presence of vulnerable plaque, data on restenosis levels etc.

During excitation the sensor transmits an electromagnetic signal, ideally an RF modulated signal, incorporating the detected information gathered by or stored in the sensor to the external electronic device 20 for signal processing.

A typical basic guidewire 1, as shown by way of examples only in the figures, is of round cross-sectional shape and can be manufactured to a range of diameters. It may incorporate a tapered section for example as in the device shown in FIG. 1, upon which a coil incorporating the sensing device or devices are mounted on its distal section.

Such a tapered section has two functions; the first is to assist in the tracking of the guidewire through the anatomical structures of the vasculature or other bodily passageway structure to the target site; the second is to provide space at the tip of the guidewire for receiving a sensor. Ideally, the sensor is placed as close to the distal tip of the guidewire as possible and this is facilitated by tapering the distal section so that it can accommodate the sensor whilst retaining the tip flexibility needed for tracking the device through tortuous bodily lumens. Desirably, the sensor on the tapered distal section advances with the leading tip of the guidewire enabling the vessel walls and physiological state to be measured and profiled for disease states, such as presence of vulnerable (soft) or calcified plaque. Gelatinous (soft) plaque will provide a different signal feedback than calcified plaque, enabling the clinician to detect that the guidewire tip has reached a target area of interest and simultaneously providing some key information as to the nature of an occlusion encountered at that target area. Other physiological variables may equally be measured by, ideally, a sensor mounted as close to the leading distal tip of the guidewire as possible.

In one arrangement, the external electronic device 20 uses SAW (surface acoustic wave) technology to analyse the data collected by the sensor. This analysis enables the clinical specialist to monitor, external to the body of the patient, desired physiological variables such as blood oxygen saturation levels, temperature, blood flow, pH and the presence of protein marker(s) inter alfa.

Temperature and flow can be determined using conventional micro QCM (quartz crystal micro-balance). In the case of biochemical species, sensing for various chemicals, biochemicals or bio-makers is realised through the attachment of selective chemical species on the surface of the device that interact and affect the mass and so the SAW properties of the device.

The device also has application in physiological diagnostic analyses. A physical “map” of the wall of the vessel at the target site may be generated to enable visualisation of the topology of the wall to be achieved. For example, a sensor can be selected which can detect and discriminate between vulnerable and calcified plaque and profile the anatomical surface structure of the vessel wall at a target or treatment site. Further it can be used to characterise the nature and extent of any restenosis that may have taken place in a vessel wall following an earlier interventional procedure such as balloon angioplasty or stent implantation.

Other applications include mapping the vascular anatomy to provide data to be used for planning revascularisation interventions; planning the repair of abdominal aortic, renal and neural lesions; assessing the condition of bypass grafts; evaluating the performance of vascular, coronary implants and other implants; and monitoring the condition of repaired aneurysms.

Technology for the characterisation of local anatomy has already been described. This uses IVUS (intravascular ultrasound) devices which allow for 2 dimensional and 3 dimensional reconstructions of the anatomy. These known devices incorporate ultrasound technology which is connected through wires at the proximal end of the device to the data acquisition system in an external device. By contrast, the present invention uses wireless EM (such as RF) technology and therefore does not require cumbersome wires connected to the external data acquisition system.

The availability of information for physicians, e.g., on the integrity of an artery (or other structure) or lesion they are treating (e.g., vulnerable gelatinous or calcified plaque in a blood vessel) is a key factor in unsuccessful interventions. Having access to this information either before or in the course of a procedure will help the physician treating the patient in settling on a treatment and/or make the physician aware of other threats. The device can be used in the assessment of: the anatomical structure (vessel or other body lumen or channel); the nature of atherosclerotic plaque; the measurement of physiological variables such as temperature; presence of specific proteins etc. All add to the information available to the physician conducting a therapeutic procedure. The device can also be used for the characterisation and assessment of a patient for clinical purposes.

Some advantages of the device of the invention include:

the device integrates sensors attached to wireless RF guidewires that can profile a vessel wall and measure physiological variables;

the device telemetry is acquired remotely without the need for wires or other forms of connectivity to the device, other than the external electronic (possibly hand-held) device;

the deliverability of the guidewire is not impaired by the incorporation of the sensor on the guidewire;

the guidewire does not require any further modification to be used as a standard guidewire and only utilises its wireless sensing technology as and when required;

the device allows for in situ characterisation of the anatomical and/or biochemical nature of irregular tissue or embolismic materials in the vasculature; and

tracking of the device through the vascular system allows for the characterisation of the healthiness of the tissue and the generation of a map of the patient's physiology and pathology.

Software in the external electronic device (which is optionally hand-held) device interrogates and interprets the data collected by and from the sensor to allow for basic binary, calcified or non calcified, vulnerable plaque, non vulnerable plaque, healthy adventitia, elevated protein and so on to be measured, and makes this ‘shorthand’ information available to the attending clinician. Bluetooth technology can be used to abstract data in real time and provide a visual image of the procedure.

The present invention provides for the following:

the integration of wireless sensor technology in a conventional guidewire construction;

the integration of surface acoustic wave wireless sensor technology in a guidewire;

the integration of conventional sensor devices with RF technology on a guidewire;

the integration of printed RFID system on a guidewire;

the provision of a micro machined guidewire for the accommodation of an integral sensor;

the integration of wireless sensor technology in the distal tip (and coil) of a guidewire;

the integration of multiple sensors in a guidewire for monitoring proteins or other biomarkers;

the integration of a wireless telemetry system with a guidewires to assist clinical decision making;

the use of surface acoustic wave technology to monitor blood flow during an angioplasty procedures;

the use of SHSAW (shear horizontal surface acoustic wave) to monitor temperature in the course of a procedure;

the use of a wireless sensor integrated in a guidewire to map vasculature; and

The use of a guidewire as a analytical diagnostic device.

Two of the main uses of the device is to monitor remotely measurements of physiological variables and/or to profile the venous or arterial anatomy. Other applications include mapping the vascular anatomy for planning revascularisation, planning the repair of abdominal aortic, renal and neural aneurysms, assessing bypass grafts, and evaluating the performance of vascular and coronary stents.

This technology can also greatly assist the interventional cardiologist in anticipating potential complications prior to intervention.

Aside from medical applications, the device of the invention also has application in non-medical fields where visualisation or diagnosis of faults in remote or inaccessible areas of channels is needed. For example, the device can be used in the assessment of cracks and occlusions in fuel line and water line systems to detect wall build-up of unwanted materials. It may also be used as a permanent implant in specific types of ducting for periodic monitoring of flow pressure etc along specific critical points.

It will of course be understood that the invention is not limited to the specific details herein described, which are given by way of example only, and that various modifications and alterations are possible within the scope of the invention, as defined by the appended claims.

1. A guidewire device comprising an elongate flexible guidewire having at least one integral wireless electromagnetic (EM) energy enabled sensor device adapted to determine a biological, physical or topographical variable.

2. A guidewire device as claimed in claim 1, in which the sensor device is radio-frequency (RF) enabled, and the guidewire comprises an antenna for transmission of wireless signals to and from the sensor device.

3. A guidewire device as claimed in claim 1, in which the sensor device is printed, etched or micro-machined onto a surface of the guidewire at or near a distal tip of the guidewire.

4. A guidewire device as claimed in claim 1, in which the sensor device includes means for determining or measuring a biological variable comprising one or more physiochemical variable, a biochemical variable, pH, blood oxygenation level, protein level, an antigen level and/or a bio-marker.

5. A guidewire device as claimed in claim 4, in which the means for determining or measuring the biological variable includes one or more analyte incorporated on or received in the guidewire.

6. A guidewire device as claimed in claim 1, in which the sensor device includes means for determining or measuring a physical variable comprising one or more of flow rate, temperature or pressure.

7. A guidewire device as claimed in claim 1, in which the sensor device includes power means for energising the sensor device in response to a remotely-generated EM signal transmitted to the sensor device by wireless means.

8. A guidewire device as claimed in claim 1, in which the sensor device includes an electronic processor having a wireless receiver for receiving a remotely-generated actuation signal and a wireless transmitter for transmitting sensed data to an external device.

9. A guidewire device as claimed in claim 8, in which the processor includes memory means for storing sensed data.

10. A guidewire device as claimed in claim 8, in which the electronic processor transforms the sensed data into a modulated form for wireless transmission to an external device.

11. A system comprising a guidewire device including an elongate flexible guidewire having at least one integral wireless electromagnetic (EM) energy enabled sensor device adapted to determine a biological, physical or topographical variable and at least one external electronic device which communicates wirelessly with the guidewire sensor device to activate the sensor device and/or to receive sensed data transmitted from the guidewire sensor device.

12. A system as claimed in claim 11, in which the external electronic device includes means for processing data received from the sensor device and for converting the data into a visual, textual or audio output.

13. A system as claimed in claim 12, in which the external electronic device uses surface acoustic wave (SAW) technology to process or convert received data.

14. A system as claimed in claim 11, in which the guidewire sensor device includes a plurality of different sensor devices adapted to determine different variables, and the external electronic device is adapted to process and/or convert data from each sensor device, individually or collectively.

15. A system as claimed in claim 14, in which the external electronic device both transmits an actuating signal to the or each sensor device and receives data from the or each sensor device.

16. A method for detecting desired data comprising:
providing a guidewire device including an elongate flexible guidewire having at least one integral wireless electromagnetic (EM) energy enabled sensor device adapted to determine a biological, physical or topographical variable;
providing an external electronic device adapted to communicate by wireless means with a guidewire sensor device on the guidewire device;
generating and transmitting a wireless electromagnetic signal from the external electronic device capable of actuating the guidewire sensor device;
receiving the generated signal at the guidewire sensor device and processing the signal to activate the sensor device; and
transmitting the sensed data by wireless means to the external electronic device.

17. A method as claimed in claim 16, including storing sensor data in a memory means of the sensor device.

18. A method as claimed in claim 16, including modulating the sensor data using the sensor device.
